(19) United States
(12) Patent Application Publication
(10) Pub. No.: US 2012/0021044 A1
(43) Pub. Date: Jan. 26, 2012

(54) Novel Cationic Lipid, A Preparation Method of the Same and A Delivery System Comprising the Same

(75) Inventors: Yu-Kyoung Oh, Seoul (KR); Min-Sung Suh, Gyeonggi-do (KR); Hye-Jeong Shin, Seoul (KR); Ga Yong Shim, Gyeonggi-do (KR)

(73) Assignees: SNU R&DB FOUNDATION, Seoul (KR); KOREA UNIVERSITY RESEARCH AND BUSINESS FOUNDATION, Seoul (KR)

(21) Appl. No.: 12/672,910

(22) PCT Filed: Aug. 20, 2008
(86) PCT No.: PCT/KR08/04819

§ 371 Date: Feb. 10, 2010

(30) Foreign Application Priority Data
Aug. 28, 2007 (KR) 10-2007-0086524

(51) Int. Cl. A61K 31/7088; A61K 9/127; A61K 47/44; A61P 35/00; A61P 19/02; A61P 9/00; A61P 5/00; C07C 237/06; C07H 21/00; B82Y 5/00

(52) U.S. Cl. 424/450; 564/160; 514/788; 536/23.1; 514/44 R; 977/773; 977/797; 977/906

(57) ABSTRACT

The present invention provides a novel cationic lipid, a preparation method of the same and a delivery system comprising the same. The cationic lipid of the present invention is used for the preparation of delivery systems of nucleic acids or physiologically active anionic proteins. The cationic lipid of the present invention can be conveniently prepared and purified by a simple process and is therefore economically highly advantageous for industrial-scale production thereof. Further, a nucleic acid or protein delivery system comprising the cationic lipid of the present invention not only significantly improves the intracellular delivery efficiency of desired nucleic acid drugs (such as DNAs, RNAs, siRNAs, antisense oligonucleotides, and nucleic acid aptamers) or anionic proteins having physiological activity, but also is usefully used to augment therapeutic efficacy of nucleic acid or protein drugs due to attenuated cytotoxicity of the delivery system.

### TECHNICAL FIELD

The present invention relates to a novel cationic lipid, a preparation method of the same and a delivery system comprising the same.

### BACKGROUND ART

With recent elucidation of medical uses of various nucleic acids such as plasmid DNAs, small interfering RNAs (siRNAs), micro RNAs and antisense oligonucleotides, a lot of importance is given to nucleic acid-delivering materials and systems for providing effective intracellular delivery of nucleic acids.

The nucleic acid delivery system for intracellular delivery of nucleic acid materials may be broadly divided into a viral vector system and a non-viral vector system.

Examples of the non-viral vector systems may include various types of formulations such as liposomes, cationic polymers, micelles, emulsions, nanoparticles, and the like. Among constituent components of these formulations, cationic lipids provide a force for electrostatic bonding with negatively charged nucleic acids and are therefore critical for the design of nucleic acid delivery systems. The cationic lipids form complex particles with negatively charged nucleic acid molecules via stable ionic bonds. Then, the resulting complex particles will be delivered into target cells for therapeutic uses and applications, for example by cell membrane fusion or cellular endocytosis.

Conventional cationic lipids were developed to have cationicity by combination of neutral fatty acid chains with amine-containing compounds such as primary amine, secondary amine, tertiary amine, or quaternary ammonium salt.

As an early form of the cationic lipid for delivery of nucleic acids, mention may be made of N-[1-(2,3-dioleyloxy)propyl]-N,N,N-triethylammonium chloride (DOTMA) which is cationic quaternary amino lipid synthesized by Felgner's group in 1987. DOTMA is used for gene transfer by formation of a cationic liposome with dioleoylphosphatidylethanolamine (DOPE) known to have cell membrane fusion-activity. DOTMA has a hydrophobic (lipophilic) moiety made up of a C₁₈-aliphatic group with a double bond and a quaternary ammonium group connected to the lipophilic group via a spacer arm with ether linker bonds. DOTMA has high gene transfer efficiency, but exhibits disadvantages such as high cytotoxicity and need for numerous and complicated synthetic processes.

In order to solve potential toxicity problems of DOTMA and to improve intracellular delivery efficiency of nucleic acids, various derivatives of DOTMA have been developed including 1,2-dimyristyloxypropyl-3-dimethylhydroxyethyl ammonium bromide (DMRIE), N-[1-(2,3-dioleyloxy)propyl]-N,N,N-trimethylammonium methyl sulfate (DOTAP), 2,3-dioleyloxy-N-[2-(sperminecarboxyamide)ethyl]-N,N-dimethyl-1-propane ammonium trifluoroacetate (DOSPA), etc.

In addition, some derivatives of cholesterol have been synthesized for delivery of nucleic acids such as DNAs, which include 3β-[N-(N′,N′-dimethylaminoethane)carbamoyl]cholesterol (DC-Chol), dimethyl-dioctadecyl ammonium bromide (DDAB), N-(α-trimethylammonioacetyl)-didodecyl-D-glutamate chloride (TMAC) and dioctadecylamidoglycylspermine (DOGS).

Depending on their structures and number of positive charges, these cationic lipids may be classified into 1) cationic lipids (such as DC-Chol, DDAB and TMAC) which provide one positive charge by the presence of tertiary or quaternary amine or hydroxyethylated quaternary amine in the head group of the lipid, and 2) cationic lipids (such as DOGS) which provide multiple positive charges by a head group of the lipid with attachment of polyamine such as spermine.

Another type of the cationic lipid used for the nucleic acid delivery is a quaternary ammonium detergent, which includes a single chain detergent such as cetrimethylammonium bromide and a double chain detergent such as dimethyldioctadecyl ammonium bromide. These detergents can deliver nucleic acids into any type of animal cells. The amine group in these amphiphiles is quaternary and a single chain of the lipid is connected to the primary amine group without the spacer arm or linker bonds. Unfortunately, pharmaceutical formulations using these amphiphilic detergents typically show significant cytotoxicity upon administration to a subject. Another type of amphiphilic molecules include 1,2-dioleoyl-3-(4′-trimethylammonio)butanoyl-sn-glycerol, cholesteryl(4′-trimethylammonino) butanoate and 1,2-dioleoyl-3-succinyl-sn-glycerol choline ester, which are structural analogues of DOTMA, but disadvantageously have low intracellular nucleic delivery efficiency.

Among the non-viral vector systems, cationic lipids provide various advantages such as easy and convenient preparation of delivery systems, low immunological side effects even after repeated administration by viral capsid proteins, no potential risk associated with in vivo safety of viral genes per se, and commercially advantageous low production costs and processes, when compared with viral gene delivery systems such as Lentivirus, Adenovirus, and the like. However, numerous cationic lipids for the nucleic acid delivery, disclosed in conventional arts, still have various disadvantages that have yet to be resolved, in terms of synthetic methods, cytotoxicity and intracellular nucleic acid delivery efficiency. To this end, there is a strong need for development of a technique which can be prepared by a short synthetic process and is capable of achieving efficient intracellular delivery of nucleic acids with low cytotoxicity.

In addition to nucleic acids, it was also pointed out that physiologically active proteins have disadvantages such as low pharmacokinetic retention time due to a short in vivo half life, need for frequently repeated administration, etc. For physiologically active proteins, there have been employed techniques to increase an in vivo retention time of the protein by chemical conjugation with a polymer material such as polyethylene glycol. However, the chemical conjugation results in chemical modification of physiologically active sites of the protein, which frequently leads to decreases in inherent physiological activity of the protein. For this reason, there is a need for development of a delivery system which is capable of preventing rapid decomposition of a physiologically active protein due to protease attack in vivo while not causing undesirable chemical modification of the protein. In particular, when heparin or the like which is a physiologically active anionic protein forms an electrostatic complex with a cationic delivery system, it is possible to alter in vivo pharmacokinetic characteristics of the target protein without chemical structural modification.

As a result of a variety of extensive and intensive studies and experiments to solve the problems as described above, the inventors of the present invention developed a method of imparting cationic properties by binding of an anionic amino acid to a fatty acid derivative having an amine structure, unlike conventional synthetic methods of cationic lipids. The present invention has been completed based on this finding.

### DISCLOSURE OF THE INVENTION

### Technical Problem

It is an object of the present invention to provide a novel cationic lipid, preparation thereof and a delivery system comprising the same. The cationic lipid of the present invention can be formulated into various types of nucleic acid delivery systems or protein delivery systems of anionic proteins having physiological activity and then can be used to enhance intracellular delivery of target materials.

### Technical Solution

The present invention provides a cationic lipid represented by Formula (I):

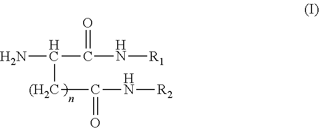

wherein:

n is 1 or 2, and

each of R₁ and R₂ is independently C₁₂-C₂₀ saturated or unsaturated hydrocarbon.

In one embodiment of the present invention, each of R₁ and R₂ may be a saturated or unsaturated hydrocarbon containing 16 carbon atoms.

In another embodiment of the present invention, each of R₁ and R₂ may be a saturated or unsaturated hydrocarbon containing 18 carbon atoms.

The cationic lipid of the present invention represented by Formula (I) is a combination of a negatively charged amino acid group with a hydrophobic C₁₂-C₂₀ saturated or unsaturated fatty acid amine derivative.

The cationic lipid of the present invention is an amphiphilic compound composed of a hydrophilic amino acid group and a hydrophobic fatty acid moiety, wherein a carboxylic group (—COOH) of the amino acid and an amine group (—NH₂) of the fatty acid derivative are connected via an amide bond.

Therefore, the present invention further provides a method for preparing a cationic lipid of Formula (I), comprising linking a carboxylic group (—COOH) of an anionic amino acid fatty acid to an amine group (—NH₂) of a fatty acid amine derivative via an amide bond.

There is no particular limit to the fatty acid amine derivative constituting the cationic lipid of the present invention, as long as it is C₁₂-C₂₀ saturated or unsaturated fatty acid. Examples of the fatty acid amine derivative may include oleylamine, myristylamine, palmitylamine, stearylamine, laurylamine, linoleylamine, arachidylamine, and the like.

The amino acid group constituting the cationic lipid of the present invention may be any amino acid having negative charge(s) and containing 10 carbon atoms or less. Preferred is glutamic acid (E) or aspartic acid (D).

A cationic lipid of Formula (I) wherein n is 1 is synthesized by combining a fatty acid derivative having an amine structure with aspartic acid. On the other hand, a cationic lipid of Formula (I) wherein n is 2 is synthesized by combining a fatty acid derivative having an amine structure with glutamic acid.

The cationic lipid of the present invention may be synthesized with a high yield by a simple process using an amino acid which is a constituent of the protein. In the cationic lipid of the present invention, amine groups of glutamic acid and aspartic acid are in positively charged forms in a neutral pH range of a normal in vivo environment, so the cationic lipid of Formula (I) will have a net positive charge in cellular environment. Positive charges of the cationic lipid enables formation of a complex with a variety of nucleic acids negatively charged in a neutral pH range and facilitate to increase contact with a target cell membrane which has relatively negative charges in vivo. Therefore, the cationic lipid of the present invention can be used in the preparation of various types of nucleic acid delivery formulations, such as liposomes, micelles, emulsions, and the like.

Therefore, the present invention further provides a nucleic acid delivery system comprising the cationic lipid of Formula (I). As used herein, the term “nucleic acid delivery system” refers to a nucleic acid delivery medium that binds to nucleic acids through the interaction with negatively charged nucleic acid sequences and then forms a complex which can be intracellularly introduced into target cells.

As used herein, “nucleic acid” is intended to encompass RNAs, small interfering RNAs (siRNAs), antisense oligonucleotides, DNAs, aptamers, and the like.

In embodiments of the present invention, a nucleic acid delivery system comprising the cationic lipid of the present invention mediates intracellular delivery of nucleic acids including RNAs, siRNAs, antisense oligonucleotides, DNAs, aptamers, and the like.

The nucleic acid delivery system of the present invention may be a formulation selected from the group consisting of liposomes, micelles, emulsions and nanoparticles.

In addition to the cationic lipid component of the present invention, the nucleic acid delivery system may further comprise a lipid derivative such as galactose-derivatized lipid, mannose-derivatized lipid, folate-derivatized lipid, PEG-derivatized lipid, or biotin-derivatized lipid.

In one embodiment of the present invention, the nucleic acid delivery system may be a liposome formulation containing the aforesaid cationic lipid and a cell-fusogenic phospholipid. Examples of the cell-fusogenic phospholipid may include dioleoylphosphatidylethanolamine (DOPE) and 1,2-diphytanoyl-sn-glycero-3-phosphoethanolamine.

In another embodiment of the present invention, the nucleic acid delivery system may be a micelle formulation containing the aforesaid cationic lipid and a surfactant. Examples of the surfactant may include Tween 20, polyethylene glycol monooleyl ether, ethylene glycol monododecyl ether, diethylene glycol monohexyl ether, trimethylhexadecyl ammonium chloride, dodecyltrimethyl ammonium bromide, cyclohexylmethyl β-D-maltoside, pentaerythritylpalmitate, lauryldimethylamine-oxide, and N-lauroylsarcosine sodium salt.

In another embodiment of the present invention, the nucleic acid delivery system may be an emulsion formulation containing the aforesaid cationic lipid and a surfactant. The surfactant that can be used in the emulsion formulation may be categorized into cationic, zwitterionic, and nonionic. Examples of the cationic surfactant may include cetyl trimethylammonium bromide, hexadecyl trimethyl ammonium bromide, and the like. Examples of the zwitterionic surfactant may include dodecyl betaine, dodecyl dimethylamine oxide, 3-(N,N-dimethylpalmitylammonio)propane sulfonate, and the like. Examples of the nonionic surfactant may include Tween 20, Tween 80, Triton X-100, polyethylene glycol monooleyl ether, triethylene glycol monododecyl ether, octyl glucoside, N-nonanoyl-N-methylglucamine, and the like.

The nucleic acid delivery system of the present invention in the form of a cationic liposome, micelle or emulsion formulation can significantly enhance delivery efficiency of the desired nucleic acids into animal cells and can also reduce the potential cytotoxicity.

The nucleic acid delivery system containing the cationic lipid of the present invention can achieve effective delivery of nucleic acids into any type of animal cells, depending upon the desired uses and applications of the nucleic acids to be transferred. The following Examples are provided to evaluate nucleic acid delivery efficiency of the nucleic acid delivery system into various types of tumor cells (the human cervical carcinoma epithelial cell line SiHa, the human lung carcinoma cell line A549, the human vaginal keratinocyte cell line VK2, and the murine hepatoma cell line Hepa1-6).

For this purpose, a complex with a variety of formulations containing the cationic lipid is formed using Block IT (Invitrogen, USA) that is a fluorescein-labeled dsRNA, and is then delivered into target cells. This is followed by examination under a fluorescence microscope to specifically measure the capacity of the cationic lipid to deliver nucleic acids into target cells. Further, the cytotoxicity of the nucleic acid delivery system in accordance with the present invention may be evaluated by using a calorimetric tetrazolium (3-(4,5-dimethylthiazol-2-yl)-2,5-diphenyltetrazolium bromide (MTT)) assay.

The nucleic acid delivery systems comprising the cationic lipid disclosed in the present invention, such as liposomes, micelles and emulsions, not only significantly increase an intracellular delivery degree but also significantly decrease the cytotoxicity, as compared to a cationic phospholipid liposome formulation containing DC-Chol which is a cationic lipid that has been conventionally used to enhance nucleic acid delivery efficiency in various cell types. Therefore, the nucleic acid delivery system of the present invention can be effectively used in therapies using nucleic acid drugs such as DNAs, RNAs, siRNAs, antisense oligonucleotides, and nucleic acid aptamers,

Further, the present invention provides a complex of the aforesaid nucleic acid delivery system with a nucleic acid. The nucleic acid delivery system of the present invention in the form of a liposome, micelle, emulsion or nanoparticle formulation is positively charged due to the presence of cationic lipid. Therefore, due to the presence of positive charges of the nucleic acid delivery system and negative charges of the nucleic acid, a complex between the nucleic acid delivery system and the nucleic acid may be formed via electrostatic bonding, by simple mixing of these two components.

The nucleic acid delivery system/nucleic acid complex may be introduced into target cells for the treatment of various diseases such as tumors, arthritis, cardiovascular diseases and endocrine diseases, which are caused by overexpression of pathogenic proteins. The nucleic acid delivery system of the present invention exhibits excellent nucleic acid delivery efficiency and low cytotoxicity, so it is possible to obtain excellent therapeutic effects by inhibiting intracellular overexpression of pathogenic proteins.

Accordingly, the present invention further provides a composition for prevention or treatment of diseases caused by intracellular overexpression of pathogenic proteins, comprising the aforesaid nucleic acid delivery system/nucleic acid complex as an active ingredient, that is a nucleic acid therapeutic agent; a use of the aforesaid nucleic acid delivery system/nucleic acid complex for the preparation of a nucleic acid therapeutic agent; and a method for treatment of a variety of diseases caused by overexpression of pathogenic proteins, comprising introducing a therapeutically effective amount of the aforesaid nucleic acid delivery system/nucleic acid complex into cells of a subject, wherein the disease includes tumors, arthritis, cardiovascular diseases, endocrine diseases, etc.

In vivo or ex vivo intracellular introduction of a desired nucleic acid by means of the nucleic acid therapeutic agent of the present invention results in a selective reduction of expression of a target protein or otherwise correction of mutations of a target gene, which makes it possible to treat diseases caused by overexpression of pathogenic proteins or mutations of the target gene.

As used herein, the term “therapeutically effective amount” refers to an amount of the nucleic acid delivery system/nucleic acid complex that is required to exert therapeutic effects on a disease of interest. As will be apparent to those skilled in the art, the effective dose of the nucleic acid delivery system/nucleic acid complex as an active drug ingredient may vary depending upon various factors such as kinds of diseases, severity of diseases, kinds of nucleic acids to be administered, kinds of dosage forms, age, weight, general health status, sex and dietary habits of patients, administration times and routes, treatment duration, and drugs such as co-administered chemotherapeutic drugs. For adults, the nucleic acid therapeutic agent may be preferably administered at a dose of 0.001 mg/kg to 100 mg/kg once a day.

Alternatively, the cationic lipid of the present invention can be used for intracellular delivery of an anionic protein, through the formation of a complex with the anionic protein instead of nucleic acid.

Further, the present invention provides a complex of the aforesaid protein delivery system with an anionic protein. Similar to formulations of the nucleic acid delivery system in accordance with the present invention, the protein delivery system may also be prepared in the form of liposome, micelle, emulsion, and nanoparticle formulations. Further, such formulations may further comprise ingredients that were exemplified to be additionally incorporated into the nucleic acid delivery system, besides the cationic lipid ingredient. The protein delivery system of the present invention is positively charged due to the presence of cationic lipid. Therefore, a complex between the delivery system and the anionic protein may be formed through electrostatic bonding due to the presence of positive charges of the delivery system and negative charges of a protein to be delivered, by simple mixing of these two components.

The protein delivery system/anionic protein complex may be introduced to improve in vivo stability and effectiveness of a physiologically active anionic protein having therapeutic efficacy on various diseases such as tumors, arthritis, cardiovascular diseases and endocrine diseases. The protein delivery system composed of the cationic lipid of the present invention can confer protease resistance to the protein partner in vivo, through the formation of a complex with the anionic protein and can also achieve improved in vivo therapeutic effects due to low cytotoxicity.

Therefore, the present invention further provides a protein therapeutic agent comprising the aforesaid protein delivery system/anionic protein complex as an active ingredient; a use of the aforesaid protein delivery system/anionic protein complex for the preparation of the protein therapeutic agent; and a method for treatment of a variety of diseases including tumors, arthritis, cardiovascular diseases and endocrine diseases, comprising introducing a therapeutically effective amount of the aforesaid protein delivery system/protein complex into cells of a subject.

Further, the cationic lipid of the present invention may be used as a component of a diagnostic kit using a nucleic acid aptamer ex vivo. For example, it may be used to diagnose the presence of a material selectively reactive with the aptamer in a sample of interest, by coating a surface of a diagnostic plate with the cationic lipid and binding the aptamer to the coating surface.

Therefore, the present invention also provides a diagnostic kit comprising a plate coated with the cationic lipid of the present invention. An aptamer may be attached to a cationic lipid-coated surface of the diagnostic kit.

### Advantageous Effects

As illustrated hereinbefore, a cationic lipid of the present invention can be conveniently prepared and purified by a simple process and is therefore economically highly advantageous for industrial-scale production thereof. Further, a nucleic acid or protein delivery system comprising the cationic lipid of the present invention not only significantly improves the intracellular delivery efficiency of desired nucleic acids drugs (such as DNAs, RNAs, siRNAs, antisense oligonucleotides, and nucleic acid aptamers) or anionic proteins having physiological activity, but also is usefully used to augment therapeutic efficacy of nucleic acid or protein drugs due to attenuated cytotoxicity of the delivery system.

### DESCRIPTION OF THE DRAWINGS

FIG. 1 shows results of 1H NMR spectrometric determinations for cationic lipid dioleoyl glutamide prepared by combination of glutamic acid and oleylamine in Example 1;

FIG. 2 shows phase-contrast micrographs (A,B) and fluorescence micrographs (C,D) illustrating intracellular delivery of dsRNA in the human lung carcinoma cell line A549, conducted using fluorescent-labeled dsRNA for a complex with a cationic liposome of Comparative Example 1 (A,C) and a liposome formulation of Example 11 containing a cationic lipid of the present invention (B,D);

FIG. 3 shows phase-contrast micrographs (A,B) and fluorescence micrographs (C,D) illustrating intracellular delivery of dsRNA in the human cervical epithelial carcinoma cell line SiHa, conducted using fluorescent-labeled dsRNA for a complex with a cationic liposome of Comparative Example 1 (A,C) and a liposome formulation of Example 13 containing a cationic phospholipid of the present invention (B,D);

FIG. 4 shows phase-contrast micrographs (B,C) and fluorescence micrographs (D,E) illustrating intracellular delivery of dsRNA in the human vaginal keratinocyte cell line VK2, conducted using fluorescent-labeled dsRNA for a complex with a cationic liposome of Comparative Example 1 (B,D) and a micelle formulation of Example 14 containing a cationic phospholipid of the present invention (C,E) (FIG. 4A: phase-contrast micrograph of non-treated VK2 cell line as a control);

FIG. 5 shows phase-contrast micrographs (A,B) and fluorescence micrographs (C,D) illustrating intracellular delivery of dsRNA in the murine hepatoma cell line Hepa1-6, conducted using fluorescent-labeled dsRNA for a complex with a cationic liposome of Comparative Example 1 (A,C) and an emulsion formulation of Example 16 containing a cationic lipid of the present invention (B,D);

FIG. 6 shows photographs illustrating comparison of transcript expression between the target gene stat3 and the glyceraldehyde-3-phosphate dehydrogenase (GAPDH) by RT-PCR assay for intracellular delivery of stat3-selective dsRNA into the human lung carcinoma cell line A549, conducted using Liposome formulations of Comparative Examples 1 and 2 (D, C) and liposome, micelle and emulsion formulations of Examples 12, 14 and 17 containing a cationic lipid of the present invention (E, F, G) (6A: non-treated cell line A549 as a control, and 6B: stat3-selective siRNA-alone treated group);

FIG. 7 shows photographs illustrating comparison of transcript expression between a target gene stat3 and glyceraldehyde-3-phosphate dehydrogenase (GAPDH) by RT-PCR assay for intracellular delivery of stat3-selective dsRNA into the human cervical carcinoma cell line HeLa, conducted using liposome formulations of Comparative Examples 1 and 2 (D, C) and liposome, emulsion and micelle formulations of Examples 11, 16 and 20 containing a cationic lipid of the present invention (E, F, G) (7A: non-treated cell line A549 as a control, and 7B: stat3-selective siRNA-alone treated group);

FIG. 8 shows photographs illustrating comparison of inhibition of target bcl-2 transcript expression by RT-PCR assay for intracellular delivery of bcl-2 antisense oligonucleotide, conducted using a target gene bcl-2-selective antisense oligonucleotide for liposome formulations of Comparative Examples 1 and 2 (D, C) and liposome, micelle and emulsion formulations of Examples 12, 15 and 17 containing a cationic lipid of the present invention (E, F, G) (8A: phase-contrast micrograph of non-treated cell line as a control, and 6B: bcl-2-selective antisense oligonucleotide-alone treated group);

FIG. 9 shows phase-contrast micrographs (B,C) and fluorescence micrographs (E,F) illustrating intracellular delivery efficiency of siRNA to the human kidney cell line 293T as inhibition of the expression of a green fluorescent protein (GFP), conducted using siRNA selectively inhibiting GFP expression for a liposome formulation of Comparative Example 2 (B,E) and a liposome formulation of Example 12 containing a cationic lipid of the present invention (C,F) (FIG. 9A: phase-contrast micrograph of the non-treated 293T cell line, and FIG. 9D: fluorescence micrograph of the non-treated 293T cell line);

FIG. 10 shows a graph illustrating cytotoxicity test results for individual complexes of dsRNA with liposome and emulsion formulations of Examples 11, 13 and 16 containing a cationic lipid of the present invention, conducted in the human lung carcinoma cell line A549;

FIG. 11 shows a graph illustrating cytotoxicity test results for individual complexes of dsRNA with liposome formulations of Examples 12, 18 and 19 containing a cationic lipid of the present invention, conducted in the human cervical carcinoma cell line SiHa; and

FIG. 12 shows graphs illustrating cytotoxicity test results for individual complexes of dsRNA with liposome, micelle and emulsion formulations of Examples 11, 14 and 17 containing a cationic lipid of the present invention, conducted in the human vaginal keratinocyte cell line VK2.

### MODE FOR INVENTION

These and other objects, advantages and features of the present invention will become apparent from the detailed embodiments given below which are made in conjunction with the following Examples. The present invention may be embodied in different forms and should not be misconstrued as being limited to the embodiments set forth herein, and those skilled in the art will appreciate that various modifications, additions and substitutions are possible without departing from the scope and spirit of the invention as disclosed in the accompanying claims. Therefore, it should be understood that the embodiments disclosed herein are provided only for illustrating the present invention and should not be construed as limiting the scope and spirit of the present invention.

### Synthesis of Novel Cationic Lipids

### Example 1

### Synthesis of Dioleoyl Flutamide

1-1) 1 equivalent (1.47 g, 10 mmol) of glutamic acid was added to 5 mL of trifluoroacetic acid and 5 mL of dichloromethane and the resulting mixture was stirred at 40° C. for 1 hour. Then, 3 equivalents (2.18 mL, 30 mmol) of SOCl₂ were slowly added dropwise to the reaction solution in an ice bath, followed by reaction at a temperature of 0 to 40° C. for 6 hours. After the reaction was complete, trifluoroacetic acid and dichloromethane were removed by concentration under reduced pressure and the reaction was confirmed by thin layer chromatography (TLC).

1-2) The reaction product obtained in Example 1-1 was dissolved in dichloromethane, and 1.5 equivalents (4.01 g, 15 mmol) of oleylamine dissolved in dichloromethane were slowly added dropwise thereto. The mixture was stirred in an ice bath for 1 hour and 3 mL of triethylamine was added dropwise thereto, followed by reaction at a temperature of 0 to 50° C. for 4 hours. After the reaction was complete, triethylamine and dichloromethane were removed by concentration under reduced pressure, and the resulting product was dissolved in ethyl acetate and then washed two times with a supersaturated sodium chloride (NaCl) solution to remove unreacted glutamic acid. A trace amount of water in ethyl acetate containing the reaction product dissolved therein was removed with magnesium chloride (MgCl₂) and the reaction was confirmed by TLC.

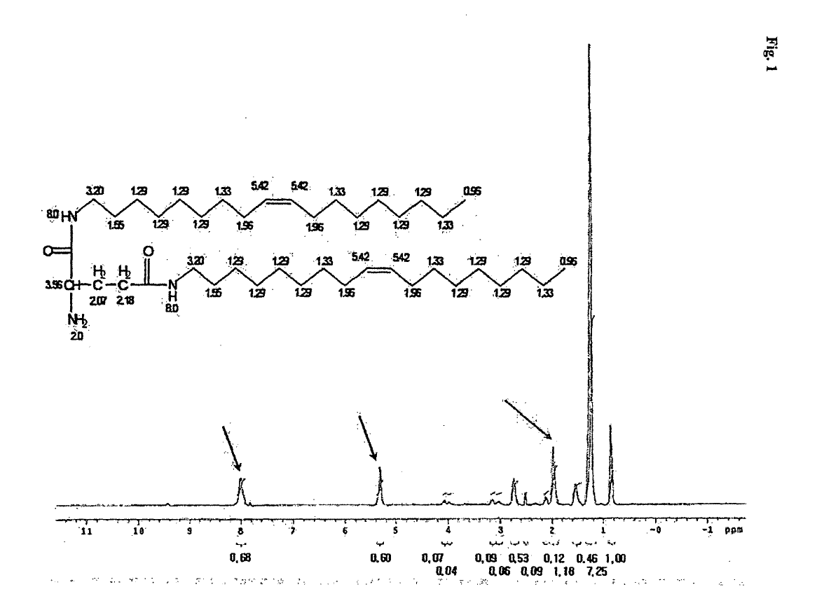

Ethyl acetate was removed by concentration under reduced pressure, and the residue was dried overnight under vacuum to thereby obtain a pale brown, highly viscous liquid product (4.12 g, yield: 92.8%). A correct structure of the final product was confirmed using a 1H NMR spectrometer. FIG. 1 shows that hydrogen atoms of an amide bond between glutamic acid and oleylamine were detected at 8.0 ppm, amine hydrogen atoms of glutamic acid were detected at 2.0 ppm, and hydrogen atoms of a characteristic double bond of oleylamine were detected at 5.42 ppm.

¹H NMR (DMSO-d₆, ppm): 8.0 (1H, —NH—CO of glutamic acid and oleylamine)

2.0 (2H, —NH₂ of glutamic acid)

5.42 (2H, —CH═CH— of oleylamine)

A reaction process of Example 1 is given in Reaction Scheme 1 below.

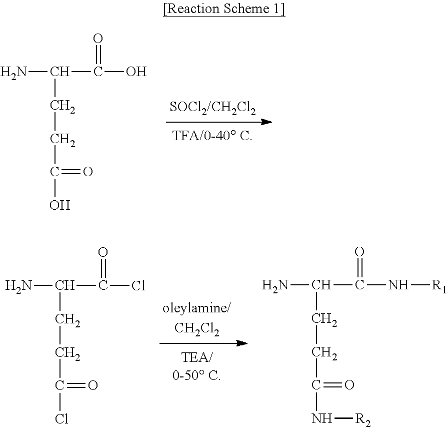

In Reaction Scheme 1, each of R₁ and R₂ is C₁₈-unsaturated (C₉) hydrocarbon.

### Example 2

### Synthesis of Dimyristoyl Glutamide

2-1) Analogously to Example 1-1, 2 equivalents of glutamic acid were reacted to obtain a glutamic acid derivative.

2-2) The reaction product obtained in Example 2-1 was dissolved in dichloromethane. Analogously to Example 1-2, the reaction was then carried out using 1.5 equivalents (3.20 g, 15 mmol) of myristylamine. A pale brown solid product (3.22 g, yield: 85.7%) was obtained and subjected to structural analysis using a ¹H NMR spectrometer.

1H NMR (DMSO-d₆, ppm): 8.0 (1H, —NH—CO of glutamic acid and myristylamine)

2.0 (2H, —NH₂ of glutamic acid)

1.29 (2H, —CH₂— of myristylamine)

A reaction process of Example 2 is given in Reaction Scheme 2 below.

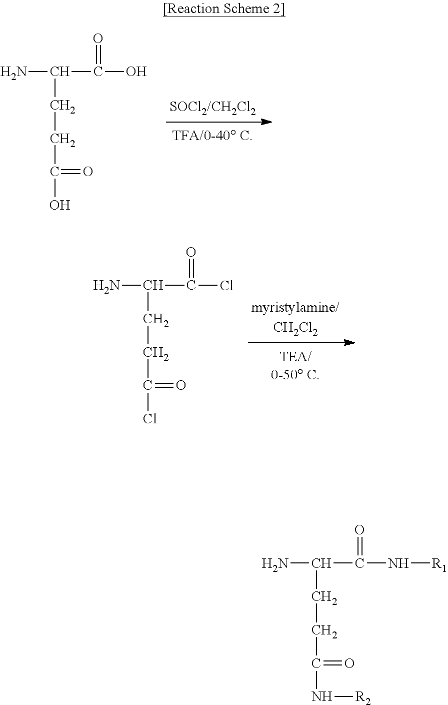

In Reaction Scheme 2, each of R₁ and R₂ is C₁₋₄-saturated hydrocarbon.

### Example 3

### Synthesis of Dipalmitoyl Glutamide

3-1) Analogously to Example 1-1, 2 equivalents of glutamic acid were reacted to obtain a glutamic acid derivative.

3-2) The reaction product obtained in Example 3-1 was dissolved in dichloromethane. Analogously to Example 1-2, the reaction was then carried out using 1.5 equivalents (3.62 g, 15 mmol) of palmitylamine. A pale brown solid product (3.74 g, yield: 90.1%) was obtained and subjected to structural analysis using a 1H NMR spectrometer.

¹H NMR (DMSO-d₆, ppm): 8.0 (1H, —NH—CO of glutamic acid and palmitylamine)

2.0 (2H, —NH₂ of glutamic acid)

1.29 (2H, —CH₂— of palmitylamine)

A reaction process of Example 3 is given in Reaction Scheme 3 below.

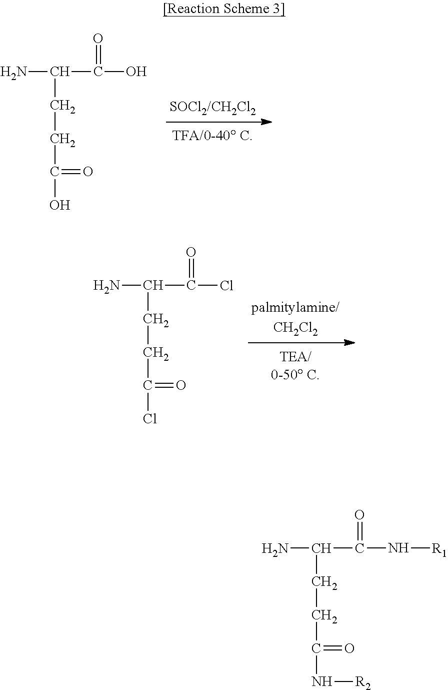

In Reaction Scheme 3, each of R₁ and R₂ is C₁₆-saturated hydrocarbon.

### Example 4

### Synthesis of Distearoyl Glutamide

4-1) Analogously to Example 1-1, 2 equivalents of glutamic acid were reacted to obtain a glutamic acid derivative.

4-2) The reaction product obtained in Example 4-1 was dissolved in dichloromethane. Analogously to Example 1-2, the reaction was then carried out using 1.5 equivalents (4.04 g, 15 mmol) of stearylamine. A pale brown solid product (3.96 g, yield: 87.1%) was obtained and subjected to structural analysis using a ¹H NMR spectrometer.

1H NMR (DMSO-d₆, ppm): 8.0 (1H, —NH—CO of glutamic acid and stearylamine)

2.0 (2H, —NH₂ of glutamic acid)

1.29 (2H, —CH₂— of stearylamine)

A reaction process of Example 4 is given in Reaction Scheme 4 below.

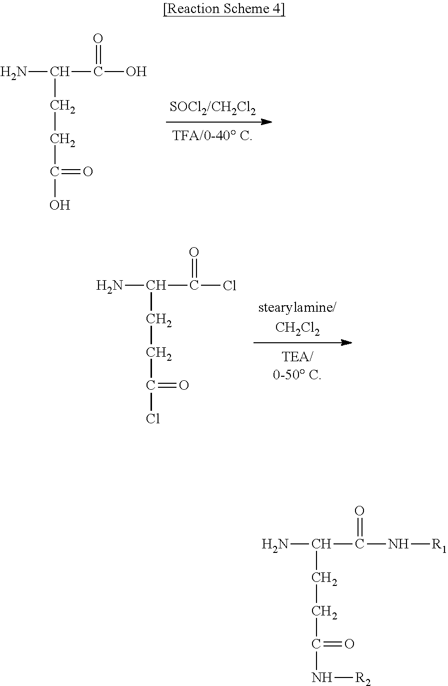

In Reaction Scheme 4, each of R₁ and R₂ is a Cᵢₛ-saturated hydrocarbon.

### Example 5

### Synthesis of Dilauroyl Glutamide

5-1) Analogously to Example 1-1, 2 equivalents of glutamic acid were reacted to obtain a glutamic acid derivative.

5-2) The reaction product obtained in Example 5-1 was dissolved in dichloromethane. Analogously to Example 1-2, the reaction was then carried out using 1.5 equivalents (2.78 g, 15 mmol) of laurylamine. A pale brown solid product (3.32 g, yield: 91.9%) was obtained and subjected to structural analysis using a 1H NMR spectrometer.

1H NMR (DMSO-d₆, ppm): 8.0 (1H, —NH—CO of glutamic acid and laurylamine)

2.0 (2H, —NH₂ of glutamic acid)

1.29 (2H, —CH₂— of laurylamine)

A reaction process of Example 5 is given in Reaction Scheme 5 below.

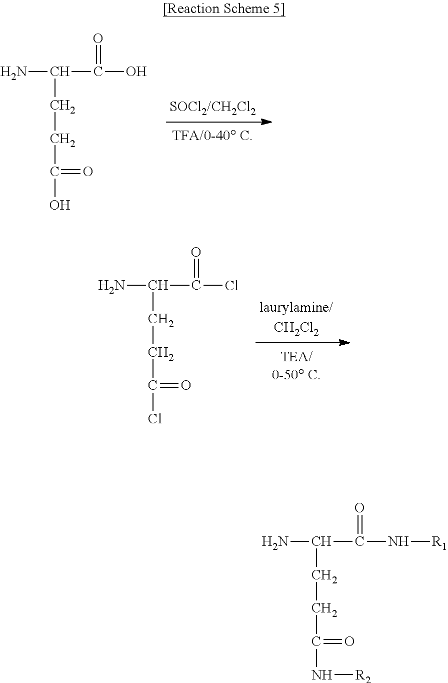

In Reaction Scheme 5, each of R₁ and R₂ is a C₁₋₂-saturated hydrocarbon.

### Example 6

### Synthesis of Dilinoleoyl Glutamide

6-1) Analogously to Example 1-1, 2 equivalents of glutamic acid were reacted to obtain a glutamic acid derivative.

6-2) The reaction product obtained in Example 6-1 was dissolved in dichloromethane. Analogously to Example 1-2, the reaction was then carried out using 1.5 equivalents (3.98 g, 15 mmol) of linoleylamine. A pale brown, highly viscous liquid product (3.72 g, yield: 82.8%) was obtained and subjected to structural analysis using a 1H NMR spectrometer.

1H NMR (DMSO-d₆, ppm): 8.0 (1H, —NH—CO of glutamic acid and linoleylamine)

2.0 (2H, —NH₂ of glutamic acid)

5.49 and 2.63 (3H, ═CH—CH₂— of linoleylamine)

A reaction process of Example 0.6 is given in Reaction Scheme 6 below.

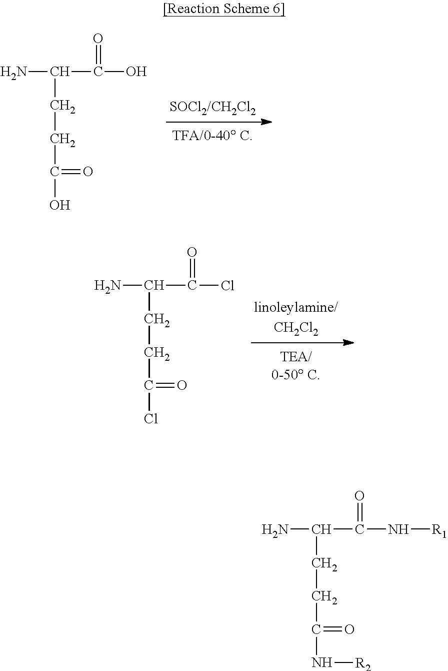

In Reaction Scheme 6, each of R₁ and R₂ is a C₁₋₈-double unsaturated (C₉,C₁₂) hydrocarbon.

### Example 7

### Synthesis of Diarachidoyl Glutamide

7-1) Analogously to Example 1-1, 2 equivalents of glutamic acid were reacted to obtain a glutamic acid derivative.

7-2) The reaction product obtained in Example 7-1 was dissolved in dichloromethane. Analogously to Example 1-2, the reaction was then carried out using 1.5 equivalents (4.46 g, 15 mmol) of arachidylamine. A pale brown solid product (3.95 g, yield: 80.2%) was obtained and subjected to structural analysis using a 1H NMR spectrometer.

1H NMR (DMSO-d₆, ppm): 8.0 (1H, —NH—CO of glutamic acid and arachidylamine)

2.0 (2H, —NH₂ of glutamic acid)

1.29 (2H, —CH₂— of arachidylamine)

A reaction process of Example 7 is given in Reaction Scheme 7 below.

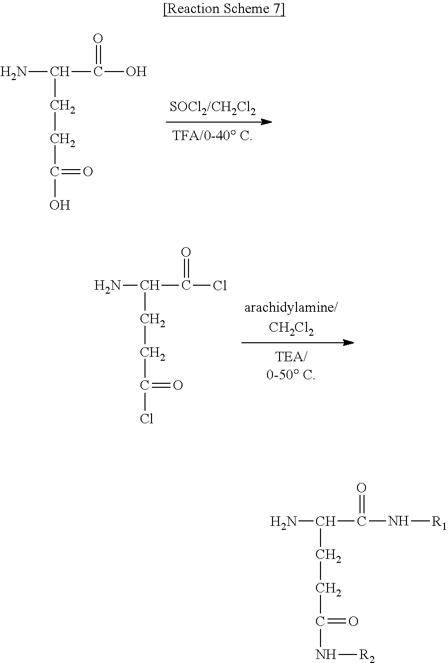

In Reaction Scheme 7, each of R₁ and R₂ is a C₂₀-saturated hydrocarbon.

### Example 8

### Synthesis of Dipalmitoyl Aspartamide

8-1) Analogously to Example 1-1, 2 equivalents of aspartic acid were reacted to obtain an aspartic acid derivative.

8-2) The reaction product obtained in Example 8-1 was dissolved in dichloromethane. Analogously to Example 1-2, the reaction was then carried out using 1.5 equivalents (3.62 g, 15 mmol) of palmitylamine. A pale brown solid product (3.59 g, yield: 88.6%) was obtained and subjected to structural analysis using a 1H NMR spectrometer.

1H NMR (DMSO-d₆, ppm): 8.0 (1H, —NH—CO— of aspartic acid and palmitylamine)

2.0 (2H, —NH₂ of aspartic acid)

1.29 (2H, —CH₂— of palmitylamine)

A reaction process of Example 8 is given in Reaction Scheme 8 below.

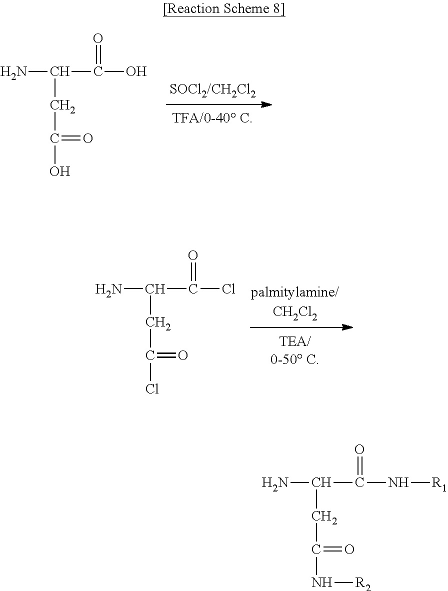

In Reaction Scheme 8, each of R₁ and R₂ is a C₁₆-saturated hydrocarbon.

### Example 9

### Synthesis of Distearoyl Aspartamide

9-1) Analogously to Example 1-1, 2 equivalents of aspartic acid were reacted to obtain an aspartic acid derivative.

9-2) The reaction product obtained in Example 9-1 was dissolved in dichloromethane. Analogously to Example 1-2, the reaction was then carried out using 1.5 equivalents (4.04 g, 15 mmol) of stearylamine. A pale brown solid product (4.02 g, yield: 90.4%) was obtained and subjected to structural analysis using a 1H NMR spectrometer.

1H NMR (DMSO-d₆, ppm): 8.0 (1H, —NH—CO— of aspartic acid and stearylamine)

2.0 (2H, —NH₂ of aspartic acid)

1.29 (2H, —CH₂— of stearylamine)

A reaction process of Example 9 is given in Reaction Scheme 9 below.

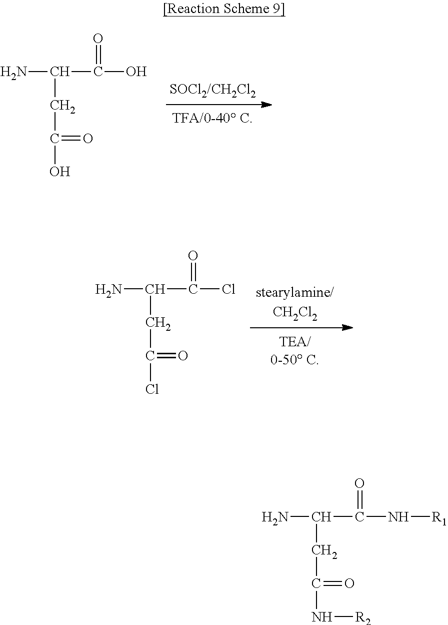

In Reaction Scheme 9, each of R₁ and R₂ is a C₁₈-saturated hydrocarbon.

### Example 10

### Synthesis of Dioleoyl Aspartamide

10-1) Analogously to Example 1-1, 2 equivalents of aspartic acid were reacted to obtain an aspartic acid derivative.

10-2) The reaction product obtained in Example 10-1 was dissolved in dichloromethane. Analogously to Example 1-2, the reaction was then carried out using 1.5 equivalents (4.01 g, 15 mmol) of oleylamine. A pale brown, highly viscous liquid product (4.07 g, yield: 92.1%) was obtained and subjected to structural analysis using a 1H NMR spectrometer.

1H NMR (DMSO-d₆, ppm): 8.0 (1H, —NH—CO— of aspartic acid and oleylamine)

2.0 (2H, —NH₂ of aspartic acid)

5.42 (2H, —CH═CH— of oleylamine)

A reaction process of Example 10 is given in Reaction Scheme 10 below.

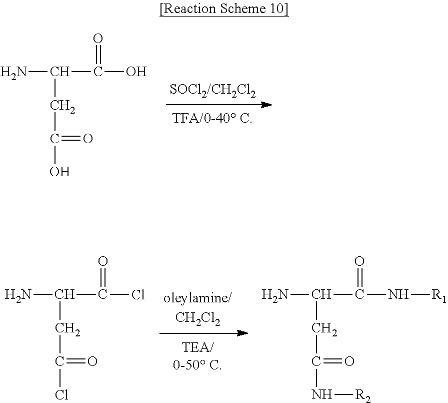

In Reaction Scheme 10, each of R₁ and R₂ is a C₁₈-unsaturated (C₉) hydrocarbon.

Preparation of Nucleic Acid Delivery Systems Containing Cationic Lipid

### Example 11

### Preparation of Cationic Liposome Containing Dioleoyl Glutamide

A cationic lipid dioleoyl glutamide prepared in Example 1 and a cell-fusogenic phospholipid DOPE (Avanti Polar Lipids Inc., USA) were each dissolved in 1 mL of chloroform. Then, each of the resulting solutions was taken in a molar ratio of 1:1, mixed in a 10 mL glass septum vial (Pyrex, USA), and then rotary-evaporated at a low speed under a nitrogen atmosphere until chloroform was completely evaporated, thereby preparing a lipid thin film. For preparation of lipid multilamellar vesicles (MLVs), 1 mL of a phosphate-buffered solution (PBS) was added to the above-prepared thin film, and the vial was then sealed at 37° C., followed by vortexing for 3 min. To obtain a uniform particle size, the vial solution was passed three times through a 0.2 μm polycarbonate membrane using an extruder (Northern Lipids Inc., Canada). The resulting cationic liposome was stored at 4° C. until use.

### Example 12

### Preparation of Cationic Liposome Containing Dimyristoyl Glutamide

A cationic lipid dimyristoyl glutamide prepared in Example 2 and a cell-fusogenic phospholipid DOPE (Avanti Polar Lipids Inc., USA) were each dissolved in 1 mL of chloroform. Then, each of the resulting solutions was taken in a molar ratio of 1:1 and mixed in a 10 mL glass septum vial (Pyrex, USA). Analogously to Example 11, a cationic liposome was prepared.

### Example 13

### Preparation of Cationic Phospholipid Liposome Containing Distearoyl Glutamide

A cationic lipid distearoyl glutamide prepared in Example 4 and a cell-fusogenic phospholipid 1,2-diphytanoyl-sn-glycero-3-phosphoethanolamine (DPhPE) (Avanti Polar Lipids Inc., USA) were each dissolved in 1 mL of chloroform. Then, each of the resulting solutions was taken in a molar ratio of 1:1 and mixed in a 10 mL glass septum vial (Pyrex, USA). Analogously to Example 11, a cationic liposome was prepared.

### Example 14

### Preparation of Cationic Micelle Containing Dimyristoyl Glutamide

A cationic lipid dimyristoyl glutamide prepared in Example 2 and a surfactant Tween 20 were taken and mixed in a molar ratio of 1:1. The resulting mixture and PBS were mixed in a ratio of 1:10 (v/v). The mixed solution was vortexed several times and then sonicated to form a cationic micelle using an ultrasonic generator for about 1 min.

### Example 15

### Preparation of Cationic Micelle Containing Diarachidoyl Glutamide

A cationic lipid diarachidoyl glutamide prepared in Example 7 and a surfactant polyethylene glycol monooleyl ether were taken and mixed in a molar ratio of 1:2. The resulting mixture and PBS were mixed in a ratio of 1:10 (v/v). The mixed solution was vortexed several times and then sonicated to form a cationic micelle using an ultrasonic generator for about 1 min.

### Example 16

### Preparation of Cationic Emulsion Containing Dipalmitoyl Aspartamide

A cationic lipid dipalmitoyl aspartamide prepared in Example 8 and Tween 80 were mixed in a molar ratio of 1:0.1. The resulting mixture and PBS were mixed in a ratio of 1:10 (v/v). The mixed solution was homogenized with a homogenizer for about 2 min to thereby prepare an oil-in-water (0/W) type cationic emulsion.

### Example 17

### Preparation of Cationic Emulsion Containing Dioleoyl Aspartamide

A cationic lipid dioleoyl aspartamide prepared in Example 10 and Tween 80 were mixed in a molar ratio of 1:0.1. The resulting mixture and PBS were mixed in a ratio of 1:10 (v/v). The mixed solution was homogenized with a homogenizer for about 2 min to thereby prepare an oil-in-water (0/W) type cationic emulsion.

### Example 18

### Preparation of Cationic Liposome Containing Dipalmitoyl Glutamide and Galactose-Derivatized Lipid

A cationic lipid dipalmitoyl glutamide prepared in Example 3, a cell-fusogenic phospholipid DPhPE (Avanti Polar Lipids Inc., USA), and a galactose-derivatized lipid cerebroside (Avanti Polar Lipids Inc., USA) were each dissolved in 1 mL of chloroform. Then, each of the resulting solutions was taken in a molar ratio of 1:1:0.05 and a cationic liposome was then prepared analogously to Example 11, thus finally obtaining a cationic liposome having galactose moieties on a surface thereof.

### Example 19

### Preparation of Cationic Phospholipid Liposome Containing Diarachidoyl Glutamide and Polyethylene Glycol (PEG)-Derivatized Lipid

A cationic lipid diarachidoyl glutamide prepared in Example 7, a cell-fusogenic phospholipid DOPE (Avanti Polar Lipids Inc., USA), and a PEG-derivatized lipid 1,2-diacyl-sn-glycero-3-phosphoethanolamine-N-[methoxy(polyethylene glycol)-3000 (Avanti Polar Lipids Inc., USA) were each dissolved in 1 mL of chloroform. Then, each of the resulting solutions was taken in a molar ratio of 1:1:0.05, and a cationic liposome was then prepared analogously to Example 11, thus finally obtaining a cationic liposome containing polyethylene glycol moieties on a surface thereof.

### Example 20

### Preparation of Cationic Phospholipid Micelle Containing Distearoyl Aspartamide and a Folate-Derivatized Lipid

A cationic lipid distearoyl aspartamide prepared in Example 9, a folate-derivatized lipid 1,2-distearoyl-sn-glycero-3-phosphoethanolamine-N-Plate(polyethylene glycol)-2000 (Avanti Polar Lipids Inc., USA), and a surfactant Tween 20 were taken and mixed in a molar ratio of 1:0.05:1. The resulting mixture and PBS were mixed in a ratio of 1:10 (v/v). The mixed solution was vortexed several times and then sonicated to form a cationic micelle using an ultrasonic generator for about 1 min.

### Comparative Example 1

### Preparation of Liposome Using Conventional Cationic Lipid

A cationic lipid DC-Chol (Avanti Polar Lipids Inc., USA) and a cell-fusogenic phospholipid DOPE (Avanti Polar Lipids Inc., USA) were each dissolved in 1 mL of chloroform. Then, each of the resulting solutions was taken in a molar ratio of 1:1, mixed in a 10 mL glass septum vial (Pyrex, USA), and then rotary-evaporated at a low speed under a nitrogen atmosphere until chloroform was completely evaporated, thereby preparing a lipid thin film. For preparation of lipid multilamellar vesicles (MLVs), 1 mL of a phosphate-buffered solution (PBS) was added to the above-prepared thin film, and the vial was then sealed at 37° C., followed by vortexing for 3 min. To obtain a uniform particle size, the vial solution was passed three times through a 0.2 μm polycarbonate membrane using an extruder (Northern Lipids Inc., Canada). The resulting cationic lipid liposome was stored at 4° C. until use.

### Comparative Example 2

### Conventional Commercially Available Cationic Liposome

LipofectAMINE 2000 (Invitrogen, USA), which is a conventional commercially available cationic liposome formulation, was purchased and used according to the manufacturer's instructions.

### Experimental Examples

### Nucleic Acid Delivery Efficiency of Cationic Lipid-Containing Nucleic Acid Delivery Systems

### Cell Culture

The human cervical carcinoma cell lines SiHa and HeLa, the human vaginal keratinocyte cell line VK2, the human lung carcinoma cell line A549, the human kidney cell line 293T, and the mouse hepatoma cell line Hepa1-6 were purchased from American Type Culture Collection (ATCC, USA). The SiHa and Hepa1-6 cell lines were cultured in Dulbecco's Modified Eagle's Medium (DMEM, Gibco, USA) containing 10% w/v fetal bovine serum (FBS, HyClone Laboratories Inc., USA) and 100 units/mL of penicillin or 100 μg/mL of streptomycin. The A549 cell line was cultured in RPMI 1640 (Gibco, USA) supplemented with 10% FBS, penicillin and streptomycin. The VK2 cell line was cultured in Keratinocyte-SFM (Gibco, USA) supplemented with 0.1 ng/mL of a recombinant human epidermal growth factor (rhEGF, Gibco, USA), 0.05 mg/mL of bovine pituitary extract (BPE, Gibco, USA) and 100 units/mL of penicillin or 100 μg/mL of streptomycin.

### Experimental Example I

### Evaluation of Nucleic Acid Delivery Efficiency Using Fluorescent-Labeled siRNA

I-1. Delivery efficiency of siRNA into A549 cell line On the day prior to the experiment, A549 cells were seeded on 24-well plates at a density of 8×10⁴ cells/well. When the cells of each plate were grown to 60% to 70% confluency, culture media of the plates were replaced with 500 μl/well of fresh media. 50 μl of a serum-free medium was added to Eppendorf tubes to which 2 μl of Block-iT (20 μmol, Invitrogen, USA) as fluorescent-labeled siRNA, and 10 μl of cationic liposomes prepared in Comparative Example 1 and Example 11 were then added. These materials were slowly pipetted, mixed and allowed to stand at room temperature for 20 min, thus resulting in formation of complexes. The thus-prepared complexes were added to the well plates, followed by cell culture in a CO₂ incubator at 37° C. for 24 hours. The A549 cell-cultured media were replaced with 500 μl/well of fresh media, and the gene transfer efficiency was examined under a fluorescence microscope.

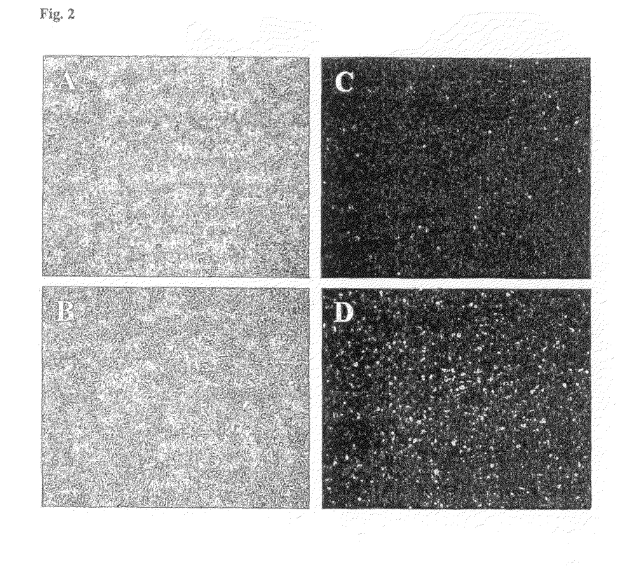

FIG. 2 shows phase-contrast and fluorescence microscopic observations illustrating nucleic acid delivery efficiency of the cationic liposomes prepared in Comparative Example 1 (A,C) and Example 11 (B,D). A: Phase-contrast microscopic image when treated with the liposome composition of Comparative Example 1. B: Phase-contrast microscopic image when treated with the liposome composition of Example 11. C: Fluorescence microscopic image illustrating intracellular delivery of fluorescent-labeled siRNA when treated with the liposome composition of Comparative Example 1. D: Fluorescence microscopic image illustrating intracellular delivery of fluorescent-labeled siRNA when treated with the liposome composition of Example 11. From the results of FIG. 2, it can be seen that the cationic liposome containing a cationic lipid of the present invention prepared in Example 11 exhibits increased siRNA delivery efficiency into A549 cells, as compared to the liposome of Comparative Example 1 with a known composition.

I-2. Delivery Efficiency of siRNA into SiHa Cell Line

On the day prior to the experiment, SiHa cells were seeded on 24-well plates at a density of 8×10⁴ cells/well. When the cells of each plate were grown to 60% to 70% confluency, culture media of the plates were replaced with 500 μl/well of fresh media. Analogously to Experimental Example I-1, each complex of Block-iT with cationic liposomes of Comparative Example 1 and Example 13 was prepared. The thus-prepared complexes were added to the well plates, followed by cell culture in a CO₂ incubator at 37° C. for 24 hours. The SiHa cell-cultured media was replaced with 500 μl/well of fresh media, and the nucleic acid delivery efficiency was examined under a fluorescence microscope.

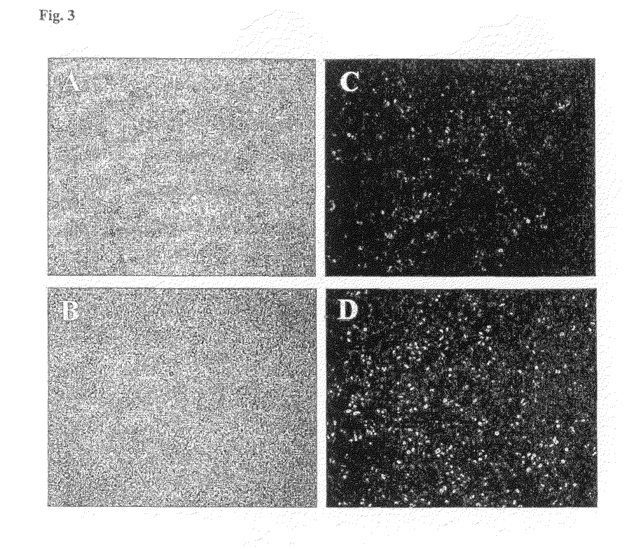

FIG. 3 shows phase-contrast and fluorescence microscopic observations illustrating nucleic acid delivery efficiency of the cationic liposomes prepared in Comparative Example 1 (A,C) and Example 13 (B,D). A: Phase-contrast microscopic image when treated with the liposome composition of Comparative Example 1. B: Phase-contrast microscopic image when treated with the liposome composition of Example 13. C: Fluorescence microscopic image illustrating intracellular delivery of fluorescent-labeled siRNA when treated with the liposome composition of Comparative Example 1. D: Fluorescence microscopic image illustrating intracellular delivery of fluorescent marker-labeled siRNA when treated with the liposome composition of Example 13. From the results of FIG. 3, it can be seen that the liposome containing a novel cationic lipid prepared in Example 13 exhibits increased siRNA delivery efficiency into SiHa cells, as compared to the liposome of Comparative Example 1 containing a known cationic lipid.

I-3. Delivery Efficiency of siRNA into Vk2 Cell Line

On the day prior to the experiment, VK2 cells were seeded on 24-well plates at a density of 8×10⁴ cells/well. When the cells of each plate were grown to 60% to 70% confluency, culture media of the plates were replaced with 500 μl/well of fresh media. Analogously to Experimental Example I-1, each complex of Block-iT with the cationic liposome of Comparative Example 1 and the cationic micelle of Example 14 was prepared. The thus-prepared complexes were added to the well plates, followed by cell culture in a CO₂ incubator at 37° C. for 24 hours. The VK2 cell-cultured media were replaced with 500 μl/well of fresh media, and the nucleic acid delivery efficiency was examined under a fluorescence microscope.

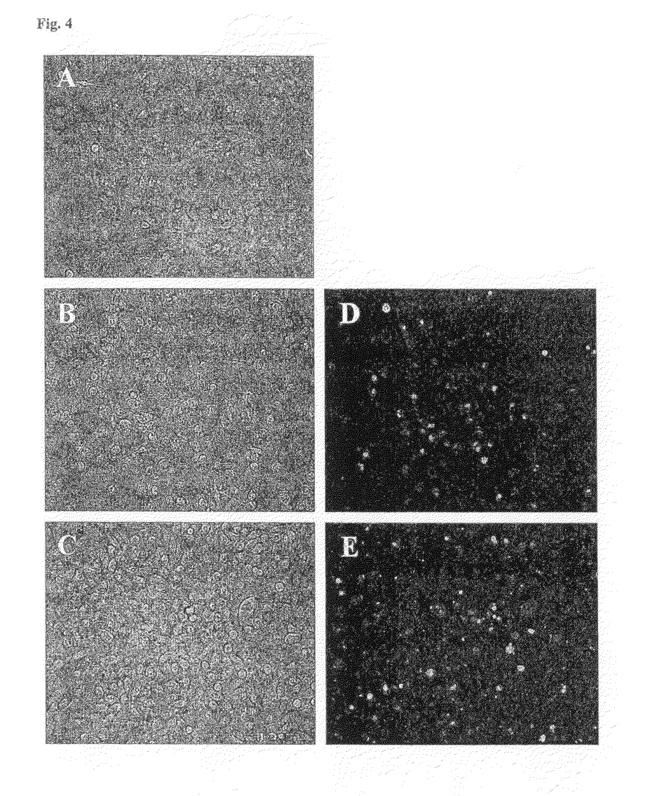

FIG. 4 shows phase-contrast and fluorescence microscopic observations illustrating nucleic acid delivery efficiency of the cationic liposome prepared in Comparative Example 1 (B,D) and the cationic phospholipid micelle prepared in Example 14 (C,E). From the results of FIG. 4, it can be seen that the cationic lipid-containing micelle prepared in Example 14 (FIG. 4E) exhibits increased siRNA delivery efficiency into VK2 cells, as compared to the liposome containing a known cationic lipid used in Comparative Example 1 (FIG. 4D). Further, as shown in FIG. 4 in terms of cell morphology observed under a phase-contrast microscope, most of cells exhibited cell shrinkage when treated with the liposome composition of Comparative Example 1 (FIG. 4B), thus representing significant deformation of cell morphology as compared to that of a non-treated control group (FIG. 4A). On the other hand, the cells treated with the cationic micelle of Example 14, as shown in FIG. 4C, exhibited the morphology similar to that of a non-treated control group (FIG. 4A), thus representing a significantly decreased cytotoxicity in terms of cell morphology.

I-4. Delivery Efficiency of siRNA into Hepa1-6 Cell Line

On the day prior to the experiment, Hepa1-6 cells were seeded on 24-well plates at a density of 8×10⁴ cells/well. When the cells of each plate were grown to 60% to 70% confluency, culture media of the plates were replaced with 500 μl/well of fresh media. Analogously to Experimental Example I-1, each complex of Block-iT with the cationic liposome of Comparative Example 1 and the cationic emulsion of Example 16 was prepared. The thus-prepared complexes were added to the well plates, followed by cell culture in a CO₂ incubator at 37° C. for 24 hours. The Hepa1-6 cell-cultured media were replaced with 500 μl/well of fresh media, and the nucleic acid delivery efficiency was examined under a fluorescence microscope.

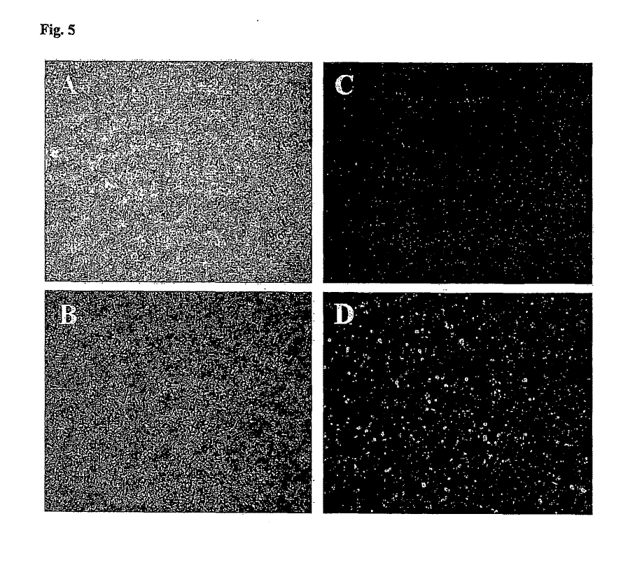

FIG. 5 shows phase-contrast and fluorescence microscopic observations illustrating nucleic acid delivery efficiency of the cationic liposome prepared in Comparative Example 1 (A,C) and the cationic phospholipid emulsion prepared in Example 16 (B,D). A: Phase-contrast microscopic image when treated with the liposome composition of Comparative Example 1. B: Phase-contrast microscopic image when treated with the cationic emulsion of Example 16. C: Fluorescence microscopic image illustrating intracellular delivery of fluorescent-labeled siRNA when treated with the liposome composition of Comparative Example 1. D: Fluorescence microscopic image illustrating intracellular delivery of fluorescent-labeled siRNA when treated with the cationic emulsion of Example 16. From the results of FIG. 5, it can be seen that the emulsion containing a novel cationic lipid prepared in Example 16 exhibits increased siRNA delivery efficiency into Hepa1-6 cells, as compared to the liposome formulation of Comparative Example 1 containing a conventional cationic lipid.

### Experimental Example II

### Evaluation of Nucleic Acid Delivery Efficiency by Identification of Gene Expression Profiles

### II-1. Delivery Efficiency of siRNA into A549 Cell Line

On the day prior to the experiment, A549 cells were seeded on 24-well plates at a density of 8×10⁴ cells/well. When the cells of each plate were grown to 60% to 70% confluency, culture media of the plates were replaced with 250 μl/well of fresh media. 50 μl of a serum-free medium was added to Eppendorf tubes to which each complex of Stat3-selective siRNA with the cationic liposome, micelle and emulsion prepared in Comparative Examples 1 and 2 and Examples 12, 14 and 17 was then added. siRNA to induce the inhibition of expression of the Stat3 gene (Genbank accession number: NM_{—}213662) was constructed using siGENOME SMARTpool (Dharmacon, Lafayette, Colo., USA). A final concentration of siRNA in the media was adjusted to 100 nM. These materials were slowly pipetted, mixed and allowed to stand at room temperature for 20 min, thus resulting in formation of complexes. The thus-prepared complexes were added to the well plates, followed by cell culture in a CO₂ incubator at 37° C. for 24 hours. After 24 hours, total RNA was isolated from the cultured cells using a Trizol reagent (Invitrogen, Carlsbad, Calif., USA) and then reverse-transcribed into cDNA using AccuPower RT PreMix (Bioneer, Daejeon, Korea). The Stat3-specific primer had a sequence of 5′-AGTTCTCCTCCACCACCAAG-3′ (left) and 5′-CCTTCTCCACCCAAGTGAAA-3′ (right), and a size of the polymerase chain reaction (PCR) product was 348 by in length. An expression level of the Stat3 transcript was assayed by determining quantitative changes of the gene expression through normalization of a band density of the Stat3-specific PCR product against a band density occurring by amplification of the GAPDH (glyceraldehyde-3-phosphate dehydrogenase) gene.

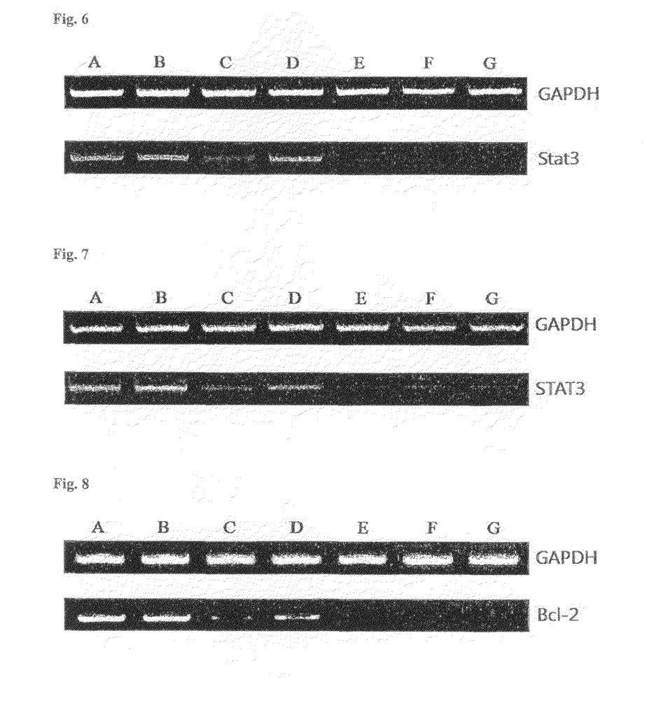

FIG. 6 shows micrographs comparing transcript expression of a target gene Stat3 in A549 cells, when the cells were treated with individual compositions. A: Control group, B: siRNA-alone treated group, C: Group treated with the composition of Comparative Example 2, D: Group treated with the composition of Comparative Example 1, E: Group treated with the composition of Example 12, F: Group treated with the composition of Example 14, and G: Group treated with the composition of Example 17. The control group (A) and the siRNA-alone treated group (B) exhibited no changes in expression of the Stat3 transcript due to no intracellular delivery of siRNA, whereas the group (D) treated with the liposome of Comparative Example 1 exhibited a less decrease in expression of the Stat3 transcript, as compared to the groups treated with the liposome, micelle and emulsion of Examples 12, 14 and 17. On the other hand, the liposome, micelle and emulsion of Examples 12, 14 and 17 exhibited efficient attenuation of Stat3 transcript expression, similar to the commercially available liposome product of Comparative Example 2. From these results of FIG. 6, it can be seen that each of the cationic lipid-containing liposome, micelle and emulsion formulations prepared in Examples 12, 14 and 17 can provide selective suppression of target protein expression via the efficient intracellular delivery of siRNA into A549 cells.

II-2. Delivery Efficiency of siRNA into HeLa Cell Line

On the day prior to the experiment, HeLa cells were seeded on 24-well plates at a density of 8×10⁴ cells/well. When the cells of each plate were grown to 60% to 70% confluency, culture media of the plates were replaced with 250 μl/well of fresh media. 50 μl of a serum-free medium was added to Eppendorf tubes to which each complex of Stat3-selective siRNA with the cationic lipid-containing liposome, emulsion and micelle prepared in Comparative Examples 1 and 2 and. Examples 11, 16 and 20 was then added. Then, experiments were carried out in the same manner and conditions as in Experimental Example II-1.

FIG. 7 shows micrographs comparing transcript expression of a target gene Stat3 in HeLa cells, when the cells were treated with individual compositions. A: Control group, B: siRNA-alone treated group, C: Group treated with the composition of Comparative Example 2, D: Group treated with the composition of Comparative Example 1, E: Group treated with the composition of Example 11, F: Group treated with the composition of Example 16, and G: Group treated with the composition of Example 20. The control group (A) and the siRNA-alone treated group (B) exhibited substantially no changes in expression of the Stat3 transcript due to low intracellular delivery efficiency of siRNA, whereas the group (D) treated with the liposome of Comparative Example 1 exhibited a less decrease in expression of the Stat3 transcript, as compared to the groups treated with the liposome, emulsion and micelle formulations of Examples 11, 16 and 20. From these results of FIG. 7, it can be seen that each of the cationic lipid-containing formulations prepared in Examples 11, 16 and 20 selectively inhibits expression of the target protein via the efficient intracellular delivery of siRNA into HeLa cells.

II-3. Delivery Efficiency of Antisense Oligonucleotide into SiHa Cell Line

On the day prior to the experiment, SiHa cells were seeded on 24-well plates at a density of 8×10⁴ cells/well. When the cells of each plate were grown to 60% to 70% confluency, culture media of the plates were replaced with 250 μl/well of fresh media. 50 μl of a serum-free medium was added to Eppendorf tubes to which each complex of an antisense oligonucleotide with the cationic lipid-containing liposome, micelle and emulsion prepared in Comparative Examples 1 and 2 and Examples 12, 15 and 17 was then added. The antisense oligonucleotide to induce the inhibition of expression of the Bcl2 gene (Genbank accession number: NM_{—}000633) was synthesized on request by Bioneer (Daejeon, Korea) (5′-TCT CCC AGC GTG CGC CAT-3′). A final concentration of the antisense oligonucleotide in the media was adjusted to 100 nM. These materials were slowly pipetted, mixed and allowed to stand at room temperature for 20 min, thus resulting in formation of complexes. The thus-prepared complexes were added to the well plates, followed by cell culture in a CO₂ incubator at 37° C. for 24 hours. After 24 hours, total RNA was isolated from the cultured cells using a Trizol reagent (Invitrogen, Carlsbad, Calif., USA) and then reverse-transcribed into cDNA using AccuPower RT PreMix (Bioneer, Daejeon, Korea). The Bcl2-specific primer had a sequence of 5′-ATG GCG CAC GCT GGG AGA AC-3′ (left) and 5′-GCG GTA GCG GCG GGA GAA GT-3′ (right), and a size of the PCR product was 348 bp in length. An expression level of the Bcl2 transcript was assayed by determining quantitative changes of the gene expression through normalization of a band density of the Bcl2-specific PCR product against a band density occurring by amplification of the GAPDH (glyceraldehyde-3-phosphate dehydrogenase) gene.

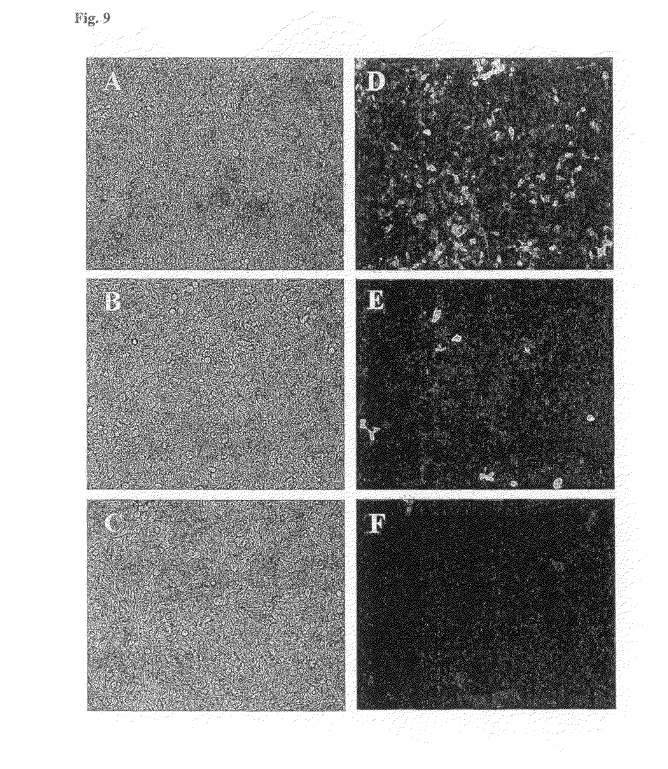

FIG. 8 shows micrographs comparing transcript expression of a target gene Bcl2 in SiHa cells, when the cells were treated with individual compositions. A: Control group, B: Bcl2-selective antisense oligonucleotide-alone treated group, C: Group treated with a complex of the antisense oligonucleotide with the liposome of Comparative Example 2, D: Group treated with a complex of the antisense oligonucleotide with the liposome of Comparative Example 1, E: Group treated with a complex of the antisense oligonucleotide with the cationic liposome of Example 12, F: Group treated with a complex of the antisense oligonucleotide with the cationic micelle of Example 15, and G: Group treated with a complex of the antisense oligonucleotide with the cationic emulsion of Example 17. The antisense oligonucleotide-alone treated group (8B) exhibited no changes in an amount of the Bcl2 transcript due to no intracellular delivery of the antisense oligonucleotide, as compared to that of a non-treated control group (FIG. 8A). The cationic lipid-containing formulations of Examples 12, 15 and 17 of the present invention exhibited effective reduction of an amount of an intracellular Bcl2 transcript, as compared to the commercially available liposome product of Comparative Example 2 (FIG. 8C) and the liposome of Comparative Example 1 (FIG. 8D). From these results of FIG. 8, it can be seen that the cationic lipid-containing formulations prepared in Examples 12, 15 and 17 effectively inhibit intracellular expression of the target protein Bcl-2 via delivery of antisense oligonucleotides into SiHa cells.

### Experimental Example III

### Evaluation of siRNA Delivery Efficiency Using Fluorescent Protein-Expressing Cell Line 293T-GFP

On the day prior to the experiment, 293T-GFP cells expressing a green fluorescent protein (GFP) were seeded on 24-well plates at a density of 8×10⁴ cells/well. When the cells of each plate were grown to 60% to 70% confluency, culture media of the plates were replaced with 500 μl/well of fresh media. 25 μl of a serum-free medium was added to Eppendorf tubes. Each complex of an siRNA inhibiting the expression of a GFP-expressing plasmid with the cationic liposomes prepared in Comparative Example 2 and Example 12 was then added to the well plates, followed by cell culture in a CO₂ incubator at 37° C. for 24 hours. The 293T-GFP cell-cultured media were replaced with 500 μl/well of fresh media, and the gene transfer efficiency was examined under a fluorescence microscope. The siRNA to induce the inhibition of GFP expression was purchased from Bioneer (Daejeon, Korea) and had a sequence of 5′-GCA UCA AGG UGA ACU UCA A-3′ (forward) and 5′-UUG AAG UUC ACC UUG AUG C-3′ (reverse). A final concentration of siRNA in the media was adjusted to 300 nM.

FIG. 9 shows phase-contrast and fluorescence microscopic observations illustrating expression of GFP in 293T cells, when the cells were treated with individual compositions. A: Phase-contrast microscopic image of non-treated GFP-expressing 293T cells. B: Phase-contrast microscopic image of 293T cells when treated with the liposome composition of Comparative Example 2. C: Phase-contrast microscopic image of 293T cells when treated with the liposome composition of Example 12. D: Fluorescence microscopic image of non-treated 293T cells. E: Fluorescence microscopic image of 293T cells when treated with the composition of Comparative Example 2. F: Fluorescence microscopic image of 293T cells when treated with the liposome composition of Example 12. Under a fluorescence microscope, fluorescent expression was clearly observed in the non-treated 293T cells due to no suppression of GFP expression. On the other hand, the cells treated with the compositions of Comparative Example 2 and Example 12 exhibited inhibition of GFP expression due to intracellular delivery of siRNA inhibiting the expression of GFP

### Experimental Example IV

### In Vivo Toxicity of Cationic Lipid-Containing Nucleic Acid Delivery Systems

IV-1. Toxicity of Cationic Lipid-Containing Nucleic Acid Delivery Systems on A549 Cell Line

The cytotoxicity of nucleic acid delivery systems containing a novel cationic lipid of the present invention was assayed according to the following experiment.

The human lung carcinoma cell line A549 was treated with each complex of siRNA with the cationic lipid-containing liposome and emulsion of Examples 11, 13 and 16 and with the siRNA gene alone, and the cytotoxicity was evaluated for individual cell groups. In order to accurately evaluate the cytotoxicity of only the nucleic acid delivery system, the siRNA used herein was scrambled RNA which is intracellularly inactive. The toxicity assay was carried out using an MTT (3-(4,5-dimethylthiazol-2-yl)-2,5-diphenyl tetrazolium bromide) colorimetric assay.

2×10⁴ cells/well of A549 cells were seeded onto 48-well plates and cultured for 12 hours. Thereafter, the cells were treated with each complex of siRNA with the cationic lipid-containing liposome and emulsion of Examples 11, 13 and 16 and with the siRNA alone. 24 hours after treatment of the cells with individual test complexes, an MTT solution was added to make 10% of the medium, followed by cell culture for another 4 hours. The supernatant was discarded and a 0.04 N isopropanol hydrochloride solution was added to the media. Then, absorbance values were measured at 570 nm using an ELISA reader. Non-treated cells were used as a control group.

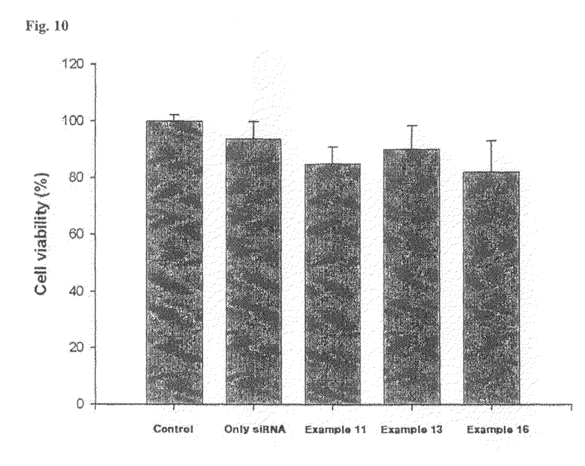

FIG. 10 shows the results of a cytotoxicity test in the human lung carcinoma cell line A549, conducted for the complexes of siRNA with the cationic lipid-containing liposome and emulsion of Examples 11, 13 and 16. As a result, the complexes of siRNA with the cationic liposome and emulsion of Examples 11, 13 and 16 exhibited no significant cytotoxicity, as compared to the control group. Therefore, it can be seen from FIG. 10 that the cationic lipid-containing liposome and emulsion formulations of the present invention prepared in Examples 11, 13 and 16 produce no significant cytotoxicity on the human lung carcinoma cell line.

IV-2. Toxicity of Cationic Lipid-Containing Nucleic Acid Delivery System on SiHa Cell Line

Analogously to Experimental Example IV-1, the human cervical carcinoma cell line SiHa was treated with each complex of siRNA with the cationic phospholipid liposomes of Examples 12, 18 and 19 and with the siRNA gene alone, and the cytotoxicity was evaluated for individual cell groups.

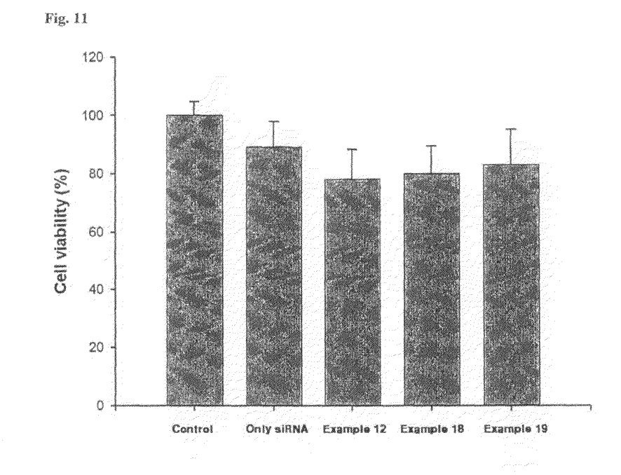

FIG. 11 shows the results of a cytotoxicity test in the SiHa cells, conducted for complexes of scrambled siRNA with the cationic lipid-containing liposomes of Examples 12, 18 and 19. As a result, the complexes of siRNA with the cationic liposomes of Examples 12, 18 and 19 exhibited no significant cytotoxicity, as compared to the control group. Therefore, it can be seen from FIG. 11 that the cationic lipid-containing liposome formulations of the present invention prepared in Examples 12, 18 and 19 produce no significant cytotoxicity on the human cervical carcinoma cancer cell line.

IV-3. Toxicity of Cationic Lipid-Containing Nucleic Acid Delivery System on VK2 Cell Line

Analogously to Experimental Example IV-1, the human vaginal keratinocyte VK2 was treated with each complex of siRNA with the cationic phospholipid liposome, micelle and emulsion of Examples 11, 14 and 17 and with the siRNA gene alone, and the cytotoxicity was evaluated for individual cell groups.

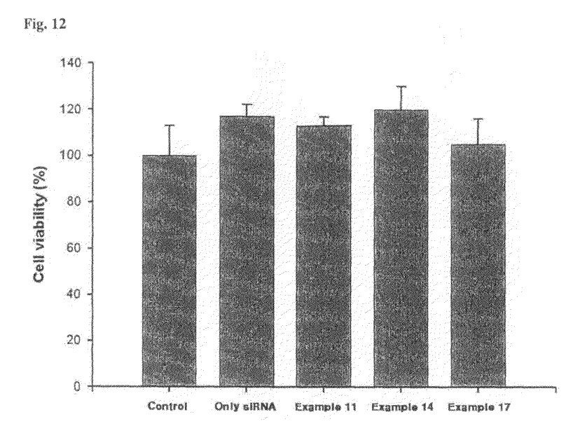

FIG. 12 shows the results of a cytotoxicity test in the human vaginal keratinocyte VK2, conducted for the complexes of scrambled siRNA with the cationic liposome, micelle and emulsion compositions of Examples 11, 14 and 17. As a result, the complexes of siRNA with the cationic liposome, micelle and emulsion of Examples 11, 14 and 17 exhibited no significant cytotoxicity, as compared to the control group. Therefore, it can be seen from FIG. 12 that the cationic lipid-containing liposome, micelle and emulsion formulations of Examples 11, 14 and 17 produce no significant cytotoxicity on the VK2 cells.

### INDUSTRIAL APPLICABILITY

As apparent from the above description, a cationic lipid of the present invention can be conveniently prepared and purified by a simple process and is therefore economically highly advantageous for industrial-scale production thereof. Further, a nucleic acid or protein delivery system comprising the cationic lipid of the present invention not only significantly improves the intracellular delivery efficiency of desired nucleic acid drugs (such as DNAs, RNAs, siRNAs, antisense oligonucleotides, and nucleic acid aptamers) or anionic proteins having physiological activity, but also is usefully used to augment therapeutic efficacy of nucleic acid or protein drugs due to attenuated cytotoxicity of the delivery system.

1. An anionic protein delivery system comprising a cationic lipid represented by Formula (I): 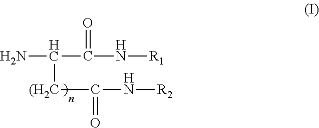
wherein:
n is 1 or 2, and
each of R₁ and R₂ is independently C₁₂-C₂₀ saturated or unsaturated hydrocarbon.

2. An oligonucleic acid delivery system comprising a cationic lipid represented by Formula (I): 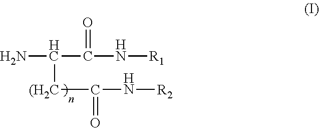
wherein:
n is 1 or 2, and
each of R₁ and R₂ is independently C₁₂-C₂₀ saturated or unsaturated hydrocarbon.

3. The oligonucleic acid delivery system of claim 2, which is for intracellular delivery of a small interfering RNA (siRNA).

4. The oligonucleic acid delivery system of claim 2, which is for intracellular delivery of an antisense oligonucleotide.

5. The oligonucleic acid delivery system of claim 2, which is for intracellular delivery of an aptamer.

6. The oligonucleic acid delivery system of claim 2, wherein the delivery system is comprised of a formulation selected from the group consisting of liposomes, micelles, emulsions, and nanoparticles.

7. The oligonucleic acid delivery system of claim 6, further comprising galactose-derivatized lipid, mannose-derivatized lipid, folate-derivatized lipid, PEG-derivatized lipid, or biotin-derivatized lipid.

8. The oligonucleic acid delivery system of claim 6, wherein the delivery system is comprised of a liposome formulation containing the cationic lipid and a cell-fusogenic phospholipid.

9. The oligonucleic acid delivery system of claim 8, wherein the cell-fusogenic phospholipid is dioleoylphosphatidylethanolamine (DOPE).

10. The oligonucleic acid delivery system of claim 8, wherein the cell-fusogenic phospholipid is 1,2-diphytanoyl-sn-glycero-3-phosphoethanolamine.

11. The oligonucleic acid delivery system of claim 6, wherein the delivery system is comprised of a micelle formulation containing the cationic lipid and a surfactant.

12. The oligonucleic acid delivery system of claim 11, wherein the surfactant is Tween 20, polyethylene glycol monooleyl ether, ethylene glycol monododecyl ether, diethylene glycol monohexyl ether, trimethylhexadecyl ammonium chloride, dodecyltrimethyl ammonium bromide, cyclohexylmethyl β-D-maltoside, pentaerythrityl palmitate, lauryldimethylamine-oxide, or N-lauroylsarcosine sodium salt.

13. The oligonucleic acid delivery system of claim 6, wherein the delivery system is comprised of an emulsion formulation containing the cationic lipid and a surfactant.

14. The oligonucleic acid delivery system of claim 13, wherein the surfactant is cetyl trimethylammonium bromide, hexadecyl trimethyl ammonium bromide, dodecyl betaine, dodecyl dimethylamine oxide, 3-(N,N-dimethylpalmitylammonio)propane sulfonate, Tween 20, Tween 80, Triton X-100, polyethylene glycol monooleyl ether, triethylene glycol monododecyl ether, octyl glucoside, or N-nonanoyl-N-methylglucamine.

15. A complex of the oligonucleic acid delivery system of claim 2 with an oligonucleotide.

16. A composition for prevention or treatment of a disease caused by overexpression of a pathogenic protein, comprising the oligonucleic acid delivery system/oligonucleotide complex of claim 15 as an active ingredient.

17. The composition of claim 16, wherein the disease is selected from the group consisting of tumor, arthritis, cardiovascular disease and endocrine disease.

18. A method for prevention or treatment of a disease caused by overexpression of a pathogenic protein, comprising administering the oligonucleic acid delivery system/oligonucleotide complex of claim 15 to a human or non-human mammal.

19. The method of claim 18, wherein the disease is selected from the group consisting of tumor, arthritis, cardiovascular disease and endocrine disease.

20. A use of the oligonucleic acid delivery system/oligonucleotide complex of claim 15 for the preparation of a therapeutic agent for a disease caused by overexpression of a pathogenic protein.

21. The use of claim 20, wherein the disease is selected from the group consisting of tumor, arthritis, cardiovascular disease and endocrine disease.
